(19) United States
(12) Patent Application Publication
(10) Pub. No.: US 2004/0208939 A1
(43) Pub. Date: Oct. 21, 2004

(54) Novel dietary compositions to reduce inflammation

(76) Inventors: Barry Sears, Swampscott, MA (US); Stacey Bell, Belmont, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/418,466

(22) Filed: Apr. 18, 2003

(51) Int. Cl. A61K 35/60; A61K 31/70

(52) U.S. Cl. 424/523; 514/22

(57) ABSTRACT

The invention relates to the use of combinations of fatty acids and specific inhibitors of delta-5 desaturase activity in a nutritional supplement to modulate the inflammatory state of a mammal. The nutritional supplement is composed of various oils selected in combination to enhance the production of anti-inflammatory eicosanoids derived from omega 6 fatty acids while simultaneously decreasing the production of pro-inflammatory eicosanoids derived from omega 6 fatty acids. In one embodiment, the invention features a combination of a fish oil, an oil containing gamma linolenic acid (GLA), and a sesame oil extract rich in one or more lignans (such as sesamol and sesamin) that are specific inhibitors of delta-5 desaturase activity.

### FIELD OF THE INVENTION

The invention relates to nutritional supplements and nutritional methods of controlling conditions related to chronic inflammation.

### BACKGROUND OF THE INVENTION

It is becoming recognized that chronic inflammation is a primary factor in the development of many disease conditions such as heart disease, cancer, and Alzheimer's disease. The balance between pro-inflammatory and anti-inflammatory eicosanoids in the body determines the ability to control chronic inflammation.

Eicosanoids are a group of hormone like substances known to play important roles in human health. Eicosanoids include such compounds as prostaglandins, leukotrienes, thromboxanes, and hydroxylated fatty acids. Yet as important as eicosanoids are, their production depends totally on the dietary intake of a specialized group of fatty acids known as essential fatty acids. Essential fatty acids cannot be made by the human body and must be supplied in the diet to provide sufficient precursors from which they can be metabolized to longer chain essential fatty acids needed to synthesize eicosanoids.

The primary essential fatty acids that are important in inflammation belong to the omega 6 family of essential fatty acids. The complexity of omega 6 essential fatty acid metabolism ultimately determines which eicosanoids are produced. This complexity is due to the activity of various enzymes responsible for the biological transformation of these essential fatty acids and how dietary factors can influence their activity, as is shown in FIG. 1. Differences in enzyme activity control the relative levels of the eicosanoid precursors dihomo gamma linolenic acid (DGLA) and arachidonic acid (AA). Eicosanoids derived from DGLA are referred to as “anti-inflammatory eicosanoids.” These same eicosanoids benefit the cardiovascular system, stimulate the immune system, and control hormone synthesis and release. On the other hand, eicosanoids derived from AA, referred to as “pro-inflammatory eicosanoids,” can inhibit cardiovascular function, depress the immune system, and are generally diameterically opposed to the physiological functions of the anti-inflammatory prostaglandins. Both pro-inflammatory and anti-inflammatory eicosanoids are necessary for proper body function, and an overabundance of either type is inconsistent with optimal physiological performance. Eicosanoids derived from the long-chain omega-3 fatty acid, eicosapentaenoic acid (EPA), are very weak inflammatory agents and thus can be considered neutral in the inflammation response. The ratio of pro-inflammatory eicosanoids to anti-inflammatory eicosanoids that are synthesized is determined by the ratio of DGLA to AA in each cell. Two enzymes, delta-6 desaturase (D6D) and delta-5 desaturase (D5D), control the ratio of DGLA to AA in each cell. The enzymes D6D and D5D are the rate limiting factors that determine the amounts of each of the prostaglandin precursors that will ultimately give rise to either anti-inflammatory eicosanoids or to pro-inflammatory eicosanoids.

### SUMMARY OF THE INVENTION

The invention features a nutritional composition for increasing the steady state ratio of anti-inflammatory eicosanoids to pro-inflammatory eicosanoids in a mammal. The composition consists essentially of, as active ingredients, an oil (usually a fish oil) containing both eicosapentaenoic acid (EPA) and docosahexaenoic acid (DHA), a source of gamma linolenic acid (GLA) that is an omega-6 fatty acid, and a specific inhibitor of delta-5 desaturase enzymatic activity. The amount of each component (i.e. the fish oil (containing EPA and DHA), GLA, and specific D5D-inhibitor) in the nutritional composition of the invention is a function of the steady state ratio of arachidonic acid (AA) to EPA in the plasma phospholipids of the mammal. Preferably, the relative and absolute amounts of each of EPA, DHA, GLA, and specific D5D-inhibitor present in the nutritional composition are effective for optimizing the ratio of anti-inflammatory to pro-inflammatory eicosanoids in the blood plasma phospholipids of a mammal to whom the nutritional composition is administered as a nutritional supplement or part of a food product.

The AA/EPA ratio is a clinical marker related to the ratio of pro-inflammatory to anti-inflammatory eicosanoids in the blood plasma phospholipids of a mammal, and is thus a reliable indicator of enhanced inflammation in the subject mammal. The desired ratio of AA/EPA requires supplementation with the invention that varies according to the particular type and extent of the existing steady state inflammatory condition of the subject mammal. Preferably, the nutritional composition of the invention is used to achieve a significantly lower AA/EPA ratio to one that is less than the AA/EPA ratio of the same subject mammal prior to being administered the nutritional supplement. For example, where the nutritional composition of the invention is administered to a mammal with an elevated AA/EPA ratio, the invention can include effective amounts of EPA, GLA, and specific D5D-inhibitor to lower the ratio of AA/EPA to less than 25, preferably less than 10, more preferably less than six, and optimally to between three and 1.5. However, the nutritional composition of the invention should not lower the AA/EPA ratio to below 0.5 as this would reduce the levels of pro-inflammatory eicosanoids that would be necessary to mount an effective challenge to infectious disease or optimal immunological response.

In one embodiment, the source of GLA in the nutritional supplement is, at least in part, an oil, for example, an oil derived from seed, such as borage, evening primrose, or black currant, an oil derived from an algae source, or an oil synthesized chemically.

As used herein, a “specific inhibitor of delta-5 desaturase,” (hereafter “specific D5D-inhibitor”) inhibits D5D activity but does not significantly inhibit D6D activity. In one embodiment, the nutritional supplement includes sesame oil, or an extract or distillate of sesame oil, as a source of at least a portion of the specific D5D-inhibitor. Preferably, the specific D5D-inhibitor is a lignan derived from sesame oil. For example, the specific D5D-inhibitor can be, without limitation, a form of a sesamin, episesamin, sesaminol, episesaminol, or sesamolin. Alternatively, the specific D5D-inhibitor can be a sesamol. In another aspect, the amounts of each of the EPA, GLA, and specific D5D-inhibitor in the nutritional supplement of the invention are further a function of the ratio of linoleic acid (LA) to GLA in a subject mammal to whom the supplement is administered. Preferably, the ratio of LA to GLA in the mammal after chronic administration of the invention is less than 100.

In yet another aspect, the amounts of each of the EPA, GLA, and specific D5D-inhibitor in the nutritional supplement of the invention are further a function of the ratio of arachidonic acid (AA) to dihomo gamma linolenic acid (DGLA) in plasma phospholipids of a subject mammal to whom the supplement is administered. Preferably, the ratio of AA to DGLA in the mammal is less than twenty in the steady state upon chronic administration of the invention.

In one embodiment, the nutritional supplement of the invention includes, in relative amounts, 1 gram of fish oil (containing 400 mg EPA and 20 mg DHA), 5 mg. of GLA, and a fraction containing specific D5D-inhibitor of at least 10 ug by weight.

The invention further features a method of increasing the ratio of anti-inflammatory eicosanoids to pro-inflammatory eicosanoids derived from omega-6 fatty acids in a mammal, the method comprising the steps of (a) administering to the mammal a nutritional supplement consisting essentially of, as active ingredients, a fish oil containing both eicosapentaenoic acid (EPA) and docosahexaenoic acid (DHA), a source of gamma linolenic acid (GLA), and a specific inhibitor of delta-5 desaturase enzymatic activity, and (b) monitoring the ratio of AA to EPA in the mammal. Preferably, monitoring the AA/EPA ratio includes obtaining a sample of blood plasma phospholipids from the mammal, and measuring the amount of AA and the amount of EPA in the sample.

The method of the invention can further include the optional step of measuring the ratio of LA to GLA of the plasma phospholipids in the mammal, wherein the LA/GLA ratio is less than 100 in the plasma phospholipids.

The method of the invention can further include the optional step of measuring the ratio of AA to DGLA in the plasma phospholipids, wherein adequate levels of a specific D5D-inhibitor are added to keep the AA/DGLA ratio less than 20 in the plasma phospholipids in the steady state upon chronic dosing with the invention.

The method of the invention can be used as a method of controlling one or more disease conditions accelerated by chronic inflammation in a mammal. Examples of inflammatory conditions that are effectively controlled by the nutritional supplement of the invention include, without limitation, cardiovascular disorders, neurological disorders, immune disorders, diabetes, and obesity.

The term “nutritional composition”, as used herein, is intended to encompass supplementation by enteral or parenteral methods and includes, without limitation, dietary supplements, pharmaceuticals, and/or medicinal supplements, in liquid or solid form, e.g., as an oil, a beverage, a tablet, a powder, or an injectable formulation, as would be known to and judged suitable by one skilled in the art. By way of example, the nutritional compositon of the invention is easily administered to a mammal by enteral administration, preferably by oral administration as a food product. The food product can be a solid or a liquid. Alternatively, the nutritional supplement is in a form conventionally known to those skilled in the art as suitable for administering oil-based vitamins and metabolites, such as, without limitation, encapsulation within a soft gelatin capsule.

In another aspect, the invention features a food product that includes the nutritional supplement of the invention in an amount effective to lower the ratio of AA/EPA in a mammal consuming such food product. Preferably, the food product contains between 1 and 80 grams, preferably between 1 and 60 grams, of carbohydrate and between 1 and 40 grams of protein. More preferably, protein and carbohydrate are present in the food product at a ratio of protein to carbohydrate between 0.5 and 1.0inclusive. The food product of the invention can include, for example, a food bar, a confectionary product, ice cream, a beverage, e.g., a ready to drink mix, a convenience food, e.g., a frozen meal, or a shelf-stable meal.

The invention further features a method of preparing the nutritional supplement of the invention, including the steps of providing a source of EPA, providing a source of GLA, and providing a source of a specific D5D-inhibitor. In one embodiment, the step of providing a source of a specific D5D-inhibitor can include providing a lignan, for example, by crystallizing the lignan from a sample of sesame oil. The lignan can be purified during crystallization using an acetone solvent.

The nutritional composition of the invention is designed to be administered to mammals for long-term control of inflammation. This is why maintaining the AA/EPA ratio of the plasma phospholipids in a desirable range is critical to the success for controlling the inflammatory response. The invention can be administered as a medicinal treatment to human patients requiring treatment for conditions relative to chronic inflammation as measured by the AA/EPA ratio. The nutritional composition of the invention may be administered to non-human mammals for veterinary or dietary purposes, or for the purpose of evaluating suitability for human administration.

### BRIEF DESCRIPTION OF THE DRAWINGS

FIG. 1 is an illustration of metabolic pathways for conversion of omega 6 essential fatty acids into pro-inflammatory and anti-inflammatory eicosanoids.

### DETAILED DESCRIPTION OF THE INVENTION

The treatment of chronic disease ultimately depends upon reducing inflammation, a physiological process that is ultimately controlled by eicosanoids. Although eicosanoids are powerful substances, they can be modulated rapidly by the nutritional composition of the invention. Success in modulating an individual's balance of eicosanoids can be monitored by specific blood tests, particularly the ratio of AA/EPA in blood plasma phospholipids.

Applicants have recognized that prior methods of enhancing the production of anti-inflammatory eicosanoids, which involved administering to a mammal combinations of an EPA-containing fish oil along with a source of GLA, suffered two disadvantages: first, that another component in the fish oil, DHA, inhibits D6D activity, and second, the unfortunate long-term conversion of GLA into AA. Applicants have thus improved upon prior EPA and GLA supplements by adding to that combination a specific inhibitor of the enzyme delta-5 desaturase, (“specific D5D-inhibitor”). The specific D5D-inhibitor is used, preferably, in the form of sesame oil, or an extract or distillate of sesame oil that is rich in one or more lignans (such as sesamol and/or sesamin). A nutritional supplement prepared from a specific combination of fish oil, an oil that contains GLA, and a specific D5D-inhibitor such as sesame oil extract, enhances production of anti-inflammatory eicosanoids derived from omega-6 fatty acids while keeping undesirable pro-inflammatory eicosanoids down to healthy levels that do not promote a chronic inflammatory response.

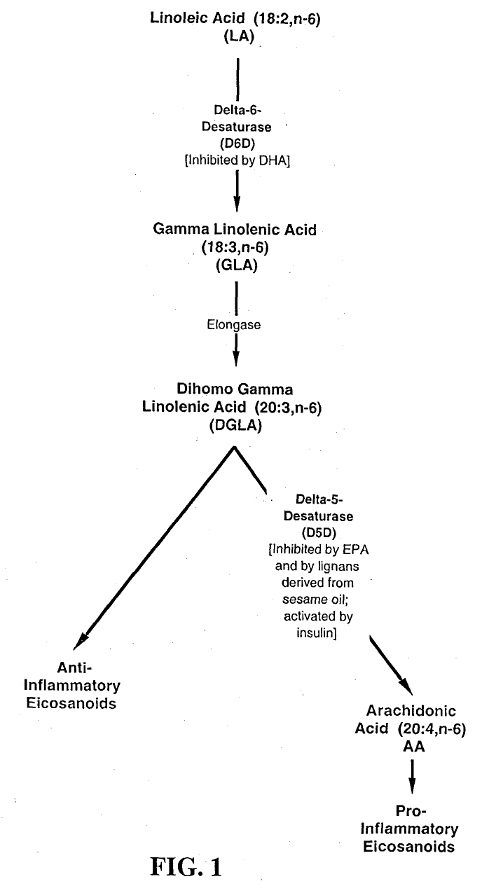

FIG. 1 illustrates the biochemical relationships of omega-6 fatty acids that are important for modulating eicosanoids. Both pro-inflammatory and anti-inflammatory eicosanoids are ultimately derived from linoleic acid (LA), an eighteen carbon atom omega-6 fatty acid that is readily accessible in the diet. However, as shown by the metabolic pathway in FIG. 1, LA can be converted into two different 20 carbon atom omega-6 fatty acids, DGLA and AA, that are the actual precurors of anti-inflammatory and pro-inflammatory eicosanoids. There are two rate-limiting enzymes in the metabolism of LA. The first is the enzyme delta-6 desaturase (D6D) that converts LA into GLA. The second is the enzyme delta-5 desaturase (D5D) that converts DGLA into AA. The rate limiting enzymes delta-6 desaturase and delta-5 desaturase control the production of various precursors of eicosanoids. The activity of these enzymes can be affected by the long-chain omega-3 fatty acids (EPA and DHA) found in fish oils. EPA acts as an inhibitor of the enzyme delta-5 desaturase, thereby inhibiting the conversion of DGLA into AA, thus reducing the AA levels in the cell membrane with a corresponding increase in EPA levels. However, the eicosanoids derived from EPA are still weak pro-inflammatory eicosanoids. DHA inhibits the delta-6 desaturase enzyme, causing LA levels to increase and levels of GLA (and thus DGLA) to decrease. The elongase enzyme that converts GLA to DGLA is not a rate limiting enzyme in these metabolic pathways and is not affected by the long-chain omega-3 fatty acids found in fish oil.

Thus fish oils, which are rich in long-chain omega-3 fatty acids, such as eicosapentaenoic acid (EPA), have the ability to moderate the overall inflammation response, but not to optimal levels. This is because the advantageous effect of EPA upon the reduction of AA levels (thus reducing the intensity of the pro-inflammatory response) is undermined by another long-chain omega-3 fatty acid found in fish oil, DHA. DHA reduces the activity of the D6D enzyme, thereby decreasing the production of GLA from LA. Decreased GLA production in turn restricts the synthesis of DGLA that is necessary for the production of many of the anti-inflammatory eicosanoids, as shown in Table 1, below.

TABLE 1
Inflammatory Eicosanoids Derived from DGLA and AA

| Anti-inflammatory Eicosanoids | Pro-inflammatory Eicosanoids |
|---|---|
| Derived from DGLA | Derived from AA |
| PGE₁ (prostaglandin E₁) | PGE₂ (prostaglandin E₂) |
| HETrE (15-hydroxyeicosatrienoic | TXA₂ (thromboxane) |
| acid) | LTB₄ (leukotriene) |
| | HETE (12-hydroxyeicosatetraenoic |
| | acid) |

GLA is produced metabolically downstream of the DHA-inhibited delta-6 desaturase, so supplementing with GLA (for example, by including a GLA-containing oil in the nutritional composition) can overcome the fish oil-induced inhibition of delta-6 desaturase. The supplementation with GLA increases the levels of DGLA that are necessary for producing anti-inflammatory eicosanoids. Unfortunately, the increased DGLA levels serves as substrate which can be converted by D5D enzyme into AA levels with chronic dosing. The increased levels of AA lead to increased levels of pro-inflammatory eicosanoids. Thus, supplementing with GLA, without additional inhibition of the D5D enzyme, can limit the potential of fish oil/GLA combinations to most effectively modulate the steady state balance of pro- and anti-inflammatory eicosanoids. What is required is an appropriate combination in which fish oil (containing EPA and DHA) and an oil containing GLA are combined with a specific D5D inhibitor. By augmenting the combination with an additional specific D5D inhibitor, the GLA supplementation is added to overcome the inhibitory effect of the fish oil on D6D, to ensure that the increased DGLA is not converted into excess AA. As a result, sufficient levels of DGLA are maintained, and this increased DGLA is ultimately metabolized into the desired anti-inflammatory eicosanoids, thus maximizing the inflammation control of the invention.

Accordingly, the invention relates to a nutritional composition, (e.g., a nutritional, dietary, or medicinal supplement), comprising ingredients formulated to reduce the levels of inflammation in a mammal. The degree of inflammation that is suitably controlled by the composition of the invention can be measured by the ratio of arachidonic acid (AA) to eicosapentaenoic acid (EPA) in the plasma phospholipids. The extraction of the plasma phospholipids from the isolated plasmalipoproteins can be performed according to the methodology described by those skilled in the art (Holub et al, “Methods in Enzymology” In: Conn PM, Means AR (eds). 141:234-244 (1987); Dewailly et al., “N-3 fatty acids and cardiovascular disease risk factors among Inuit of Nunavik,” Am J Clin Nutr 74:464-473 (2001).

The AA/EPA ratio in the plasma phospho lipids is a precise marker of inflammation, because AA is the essential fatty acid precursor to virtually all pro-inflammatory eicosanoids, whereas eicosanoids derived from EPA have little, if any, pro-inflammatory action. At the same time, the ratio of LA/GLA indicates the extent of the inhibition of delta-6 desaturase activity, e.g., by fish oil, with a high ratio of LA to GLA indicating significant inhibition of delta-6 desaturase activity. Such inhibition of D6D activity will reduce the levels of DGLA. Although the fish oil inhibition of delta-6 desaturase activity can be potentially overcome by addition of oils containing GLA (see U.S. Pat. No. 5,059,622, hereby incorporated by reference) different ratios of GLA to fish oil can have potentially adverse reactions because of the long-term conversion of the added GLA into AA.

To minimize this possibility, it would be useful to include specific inhibitors of delta-5 desaturase in the supplement formulation so that increased amounts of GLA in the formulation can be added and converted metabolically into DGLA, so that a greater number of anti-inflammatory eicosanoids can be produced without overproducing AA. The ratio of AA/DGLA of the blood plasma phospholipids indicates the extent of activity versus inhibition of the blood delta-5 desaturase activity, with a low ratio of AA to DGLA indicating significant inhibition of delta-5 desaturase activity.

The formulation of the nutritional composition of the invention can be adjusted, e.g., customized or optimized, to the physiological needs or requirements of the particular mammal (or particular cohort of mammals exhibiting similar characteristics) for whom the nutritional supplement is intended to be administered. In such cases, an optimal ratio between EPA (e.g., EPA-containing fish oil), GLA,( e.g., GLA-containing oil), and specific D5D-inhibitor is established in the nutritional composition of the invention so as to maintain the AA/EPA ratio within a range consistent with a steady state control of inflammation in the body. The optimal ratio between EPA-containing fish oil, GLA-containing oil, and specific D5D-inhibitor is further established so that the resulting plasma ratio of LA to GLA (LA/GLA) is not increased above the initial baseline, thereby avoiding the depletion of GLA and ultimately DGLA that is needed for the formation of anti-inflammatory eicosanoids. The optimal ratio between EPA-containing fish oil, GLA-containing oil, and specific D5D-inhibitor is yet further established so that the resulting plasma ratio of AA to DGLA (AA/DGLA) is optimized. The nutritional composition of the invention, to be successful, should be used on a regular (i.e. chronic) basis to maintain the steady state ratios of the AA/EPA and LA/GLA necessary to modulate chronic inflammation in the patient or animal on a long-term basis.

Alternatively, where the nutritional composition of the invention is intended for general consumption by, or adminstration to mammals, exhibiting a diverse range of starting blood AA/EPA ratios, the ratios of fish oil (containing EPA and DHA), GLA, and specific D5D-inhibitor can be set in a standardized formulation. Plasma phospholipid ratios of AA/EPA should be monitored to adjust the amount, or dosage, of the formulation consumed by any particular individual.

Preparation of a Nutritional Supplement:

The preferred physical forms of the EPA, DHA, GLA, and specific D5D-inhibitor are triglycerides. Other acceptable forms are methyl or ethyl esters, monoglycerides, free fatty acids, or the appropriate salts of free fatty acids.

Sources of EPA, DHA, GLA, and specific D5D-inhibitor include the following:

EPA and DHA: Fish oil containing both EPA and DHA can be easily extracted from natural sources such as plankton, krill, or marine animals. EPA and DHA can also be fermented under controlled conditions. In both cases, the extracted oil should be refined to meet all international standards for edible oils. The triglyceride form of fish oil can be altered either by chemical or biochemical means to produce free fatty acids, salts of free fatty acids, methyl or ethyl esters, or monoglycerides which can be further fractionated to give higher contents of EPA and DHA than the starting oil in the triglyceride form. Since the amount of EPA in fish oils required to modulate the AA/EPA is relatively high, this necessitates a high standard of fish oil purity. EPA and DHA can also be chemically synthesized.

A good starting dose of fish oil for most patients is 0.5, preferably 2.5, grams per day of long-chain omega-3 fatty acids. However, the desired AA/EPA ratio indicates the level fish oil in the composition of the invention the mammal will require. It often requires about 5-8 grams per day of long-chain omega-3 fatty acids to reduce the AA/EPA ratio in humans to the level found in the Japanese population (a AA/EPA ratio of approximately 1.5). It should be noted that the greater the degree of insulin control by the patient, the less fish oil is required. This is because any reduction in insulin levels will decrease the production of AA since insulin is an activator of the D5D enzyme (see FIG. 1). Conversely, the less the insulin control, the greater the levels of supplementation with fish oil are required to lower the AA/EPA ratio into the desired range between 1.5 to 3.

Measurement of the AA/EPA ratio from the plasma phospholipid has been shown to be superior to other blood sources such as red blood cells. First, the fatty acids in the plasma lipoprotein are in dynamic interaction with the lymphatic circulation, whereas red blood cell membranes are not. Second, the absence of hemoglobin in the lipoprotein fractions reduces the likelihood of auto-oxidation of the highly polyunsaturated long-chain omega-3 fatty acids. Third, the ratio of AA/EPA in the plasma phospholipids is different than in the red blood cells. GLA: Natural sources of GLA include a number of seed sources such as borage, evening primrose, and black currant. Like EPA, GLA can also be made by fermentation with selected algae or bacterial strains. GLA in the triglyceride form can be most easily extracted and refined from vegetable seed sources using standard technology common in the edible oil industry to create an oil suitable for human consumption as defined by international standards. Common sources of GLA would include borage, black current, evening primrose seeds and oat bran. Certain microorganisms can also be fermented to produce GLA in the triglyceride form which can likewise be refined to meet international standards established for an edible oil.

GLA isolated in the triglyceride form can be chemically or biochemically transformed into free fatty acids, salts of free fatty acids, methyl or ethyl esters, or monoglycerides which can be further fractionated by standard techniques into fractions with higher GLA content than found in the starting oils. Finally, GLA can be chemically synthesized by standard chemical techniques.

Specific D5D-inhibitor: Specific D5D-inhibitors useful in the invention include, for example, an extract of sesame oil that is rich in one or more lignins such as, without limitation, sesamol, sesimine, sesamin, episesamin, sesaminol, episesaminol, and sesamolin.

Forms of sesame oil useful as a source of specific D5D-inhibitor include crude or refined sesame oil, prepared by methods known to those skilled in the art of nutritional supplements. Also useful in the invention are distillates of sesame oil, extracts, and lignan fractions of sesame oil crystallized from acetone.

Steam distillation of refined sesame oil provides a distillate enriched in lignans. Sesame oil distillates can be prepared by subjecting crude or refined sesame oil to steam sparging at high vacuum (<0.5 mmHg) and 300-350° F. to strip off the unsaponifiable fraction. The distillate is then recovered in a condenser placed immediately after the stripper.

Unsaponifiables rich in lignans can be recovered from sesame oil distillate. Acetone fractionation of the sesame oil distillate can further enrich the lignans. Further enrichment of the lignans can be achieved by crystallization from acetone. Alternatively, enzymatic hydrolysis of the remaining triglycerides in the distillate can be used to reduce the triglycerides to free fatty acids and glycerol. The enzymatic digestion process is then followed by molecular distillation to remove the fatty acids and glycerol leaving a fraction rich in lignans. The unsaponifiable fraction of sesame oil will be rich in lignan compounds including, without limitation, sesamin, sesamol, sesamolin, sesaminol, episesaminol and episesamin.

The effect of the composition of the invention on the inflammatory response can be measured by ratios of certain key fatty acids in the isolated plasma phospholipids in the blood. The plasma phospholipids represent a steady state accumulation of essential fatty acids from the diet and thus can guide the appropriate combinations for the invention.

The combination of fish oil (containing EPA and DHA) and GLA-containing oil (such as, for example, borage oil, evening primrose oil, or black currant oil) that is required to modulate the inflammatory response should preferably have a ratio of EPA to GLA that is sufficient to ensure that the resulting steady state ratio of AA/EPA in the plasma phospholipids is decreased below baseline, but remains greater than 0.5. At too low an AA/EPA ratio in the blood, the ability of the patient or animal to fight off infection by mounting an appropriate inflammatory response may be compromised. The desired AA/EPA ratios are detailed in Table 2.

TABLE 2

| | | | | Ideal | |
|---|---|---|---|---|---|
| | Chronic | Elevated | Adequate | state of | Reduced |
| | inflamma- | inflamma- | level of | inflamma- | Inflammatory |
| Test | tion | tion | inflammation | tion | Response |
| AA/ | 15 or | 10 | 3 | 1.5 | 0.5 |
| EPA | greater | | | | |

The ideal AA/EPA ratio of 1.5 is that found in the Japanese who are considered the longest-lived and healthiest population in the world. They also have the world's lowest incidence of cardiovascular disease and depression. In contrast, the average AA/EPA of Americans is approximately 11, and for patients with inflammatory conditions and neurological disorders, the AA/EPA ratio is in excess of 20.

Preferably, the ratio of LA/GLA in the plasma phospholipids is less than 100 but greater than 5, in order to maintain adequate levels of GLA to supply the building blocks for the production of anti-inflammatory eicosanoids derived from DGLA necessary to modulate the activities of the pro-inflammatory eicosanoids derived from AA. To maintain these ratios in the plasma phospholipids using combinations of fish oil and GLA, it is necessary to add additional specific D5D-inhibitors in order to prevent any accumulation of AA that leads to increased inflammation. The amount of specific D5D-inhibitor used should be at sufficient levels in order to achieve the steady state target ratios of AA/EPA and LA/GLA in the plasma phospholipids should be optimized. A decreased AA/DGLA ratio ensures that adequate levels of DGLA are present to make sufficient amounts of anti-inflammatory eicosanoids.

The plasma phospholipid ratio of AA/DGLA (“delta-5 desaturation index”) ratio is preferably less than 20, more preferably less than 10, and most preferably three or below. For further guidance on the delta-5 desaturation index, see Fujiyama-Fujiwara et al., J. Nutr Sci Vitaminol, 41(2):217-25 (1995) and Umeda-Sawada et al., Bio Sci Biotechnol Biochem, 59 (12):2268-73 (1995).

Method of Monitoring Inflammation

Clinical monitoring of plasma phospholipids: The level of essential fatty acids can be measured in blood plasma phospholipids by drawing a blood sample from a subject mammal. The plasma is separated by centrifugation from the cellular components of the blood sample. The plasma is then extracted with a 2:1 ratio of chloroform to methanol in a ratio of one part serum to four parts of the methanol-chloroform mixture, and the lower phase containing the lipids is separated from the upper phase by methods known to those skilled in the art.

To the total lipid extract from the plasma a known amount of diheptadecanoyl phosphatidylcholine (di 17:0 PC), used as the internal standard and the lipid extract is taken to dryness under nitrogen. The dried lipids are separated on a silica gel thin layer plate and the phospholipids from the other lipid components in the lipid extract by chromatography using a heptane-isopropyl ether-acetic acid (60:40:3) solvent system.

The area of the thin layer plate containing the phospholipids are removed from the plate and extracted with methanol. The component fatty acids are methylated with boron trichloride in methanol.

Following extraction, the phospholipids are analyzed by a gas chromatograph to determine the lipid profile of the phospholipids. From that lipid profile, the ratios of AA/EPA, LA/GLA, and AA/DGLA can be determined. The integrated areas under the curve of those peaks known to correspond to LA, GLA, DGLA, AA, and EPA are calculated by standard procedures known to those skilled in the art. The ratio of the integrated areas of the appropriate peaks calculated to determine the appropriate fatty acid ratios in the isolated plasma phospholipids.

The nutritional compositon of the invention can be administered to a mammal in a manner known to those skilled in the art, including enteral and parenteral administration. Without limitation, the nutritional supplement of the invention is preferably administered by enteral means, and, more preferably, is administered by oral ingestion.

Use of the Nutritional Supplement in a Food Product:

As seen from FIG. 1, the hormone insulin can also activate the D5D enzyme. Therefore if the nutritional supplement of the invention is to be incorporated into a food format, then the ratio of protein-to-carbohydrate in such a food format is critical to maximize its full potential. This is because, at a low protein-to-carbohydrate ratio, excess insulin can be generated, and the increased insulin can stimulate the D5D activity to produce more AA, thereby increasing the inflammatory response. The ideal ratio of protein-to-carbohydrate for a food product that incorporates the nutritional composition of this invention is between 0.5 and 1.0, inclusive, and the carbohydrate content should ideally have a low glycemic index for maximum insulin control.

The composition of this invention can be included in a wide variety of formats including liquids, soft gelatin capsules, dry microcapsules, food bars, ready to drink mixes, ice creams, margarines, and other food formats into which oils or dry microencapsulated oils can be easily incorporated.

### EXAMPLE 1

Forty (40) kg. of refined sesame oil is distilled to provide 800 ml of distillate containing lignans from sesame oil. 20 ml of distillate is added to 100 ml of acetone and cooled to −8° C. The solution is filtered to provide 5 ml of fractionated distillate rich in lignans.

### EXAMPLE 2

A nutritional supplement can be prepared by combining 100 ml of a fish oil concentrate (containing 40% of the total fatty acids as EPA and 20% as DHA) with 5 ml of borage oil containing 20% GLA of the total fatty acids. The ratio of combined long-chain omega-3 fatty acids (EPA and DHA) to GLA in the resulting mixture is 60:1. To this mixture is added 5 mg. of the fractionated lignan-rich distillate in Example 1.

### EXAMPLE 3

The composition in Example 2 can, optionally, be encapsulated in a soft gelatin capsule suitable for enteral administration.

### EXAMPLE 4

The composition in Example 2 can be micro-encapsulated as a dry powder and be incorporated into a food bar containing 14 grams of protein and 20 grams of carbohydrate and 7 grams of fat. For example, the nutritional supplement can be added to a commercially available conventional food bar, for example, the food product, e.g., OmegaZone™ (Sears Labs, Danvers, Mass.). Alternatively, the supplement of the invention can be added to one or more of the food products disclosed in U.S. Publ. Appln. No. US 2001/0022980 A1, published Sep. 20, 2001, and in U.S. Pat. No. 5,902,797, issued May 11, 1999.

### EXAMPLE 5

A blood sample of 100 microliters is drawn from the patient. Plasma is separated from the cellular components of the blood by centrifugation. One ml. of plasma is extracted with 4 ml. 2:1 ratio of chloroform to methanol, and the lower phase and a known amount of diC17:0 PC added and the total lipid extract is taken to dryness under nitrogen. The dried lipids are separated on a silica gel thin layer plate to separate the phospholipids from the other lipid components of the extracted plasma using a solvent system of heptane-isopropyl ether-acetic acid (60:40:3). The phospholipids are then extracted from the silica gel and converted to the methyl esters using a mixture of BF₃ in methanol. The methyl esters are then analyzed by a gas chromatograph to determine the lipid profile of the plasma phospholipids. From that areas under the integrated curve of the fatty acid profile, the ratios of AA/EPA, LA/GLA, and AA/DGLA are determined.

While this invention has been particularly shown and described with references to preferred embodiments thereof, it will be understood by those skilled in the art that various changes in form and details may be made therein without departing from the spirit and scope of the invention as defined by the claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A nutritional supplement for increasing the ratio of anti-inflammatory eicosanoids to pro-inflammatory eicosanoids derived from omega-6 fatty acids in a mammal, said supplement consisting essentially of, as active ingredients;
(a) fish oil comprising eicosapentaenoic acid (EPA) and docosahexaenoic acid (DHA);
(b) gamma linolenic acid (GLA); and
(c) a specific inhibitor of delta-5 desaturase enzymatic activity, wherein the amount of each of said fish oil, GLA, and inhibitor in said supplement is a function of the ratio of arachidonic acid (AA) to EPA in the plasma phospholipids in said mammal.

2. The nutritional supplement of claim 1, wherein said nutritional supplement comprises sesame oil as a source of at least a portion of the specific inhibitor of delta-5 desaturase enzymatic activity.

3. The nutritional supplement of claim 1, wherein the specific inhibitor of delta-5 desaturase is a lignan.

4. The nutritional supplement of claim 1, wherein the specific inhibitor of delta-5 desaturase is sesamin.

5. The nutritional supplement of claim 1, wherein the specific inhibitor of delta-5 desaturase is episesamin.

6. The nutritional supplement of claim 1, wherein the specific inhibitor of delta-5 desaturase is sesamol.

7. The nutritional supplement of claim 1, wherein the specific inhibitor of delta-5 desaturase is sesaminol.

8. The nutritional supplement of claim 1, wherein the specific inhibitor of delta-5 desaturase is episesaminol.

9. The nutritional supplement of claim 1, wherein the specific inhibitor of delta-5 desaturase is sesamolin.

10. The nutritional supplement of claim 1, wherein said nutritional supplement comprises an oil as a source of at least a portion of said GLA.

11. The nutritional supplement of claim 10, wherein the oil is an oil selected from the group consisting of borage oil, evening primrose oil, and black currant oil.

12. The nutritional supplement of claim 1, wherein said ratio of arachidonic acid AA to EPA is less than ten.

13. The nutritional supplement of claim 12, wherein said ratio of arachidonic acid AA to EPA is less than six.

14. The nutritional supplement of claim 12, wherein said ratio of arachidonic acid AA to EPA is greater than 0.5.

15. The nutritional supplement of claim 1, wherein the amounts of each of said EPA, GLA and inhibitor in said supplement are further a function of the ratio of linoleic acid (LA) to GLA in said mammal.

16. The nutritional supplement of claim 1, wherein said ratio of linoleic acid (LA) to GLA in said mammal is less than 100.

17. The nutritional supplement of claim 1, wherein the amounts of each of said EPA, GLA and inhibitor in said supplement are further a function of the ratio of arachidonic acid (AA) to dihomo gamma linolenic acid (DGLA) in said mammal.

18. The nutritional supplement of claim 17, wherein said ratio of AA to DGLA in said mammal is less than ten.

19. The nutritional supplement of claim 1, wherein the concentration of said specific inhibitor of delta-5 desaturase in said nutritional supplement is adequate to maintain the ratio of AA/DGLA to be less than 20.

20. A method of increasing the ratio of anti-inflammatory eicosanoids to pro-inflammatory eicosanoids derived from omega-6 fatty acids in a mammal, said method comprising the steps of:
(a) administering to said mammal the nutritional supplement of claim 1; and
(b) monitoring the ratio of arachidonic acid (AA) to EPA in said mammal.

21. The method of claim 20, wherein said monitoring comprises obtaining a sample of plasma phospholipids from said mammal, and measuring the amount of AA and the amount of EPA in said sample.

22. The method of claim 20, wherein said method is used to control a cardiovascular disorder in said mammal.

23. The method of claim 20, wherein said method is used to control a neurological disorder in said mammal.

24. The method of claim 20, wherein said method is used to control an immune disorder in said mammal.

25. The method of claim 20, wherein said method is used to control a diabetic condition in said mammal.

26. The method of claim 20, wherein said method is used to control inflammation caused by obesity said mammal.

27. The method of claim 20, wherein said method is used to control obesity in said mammal.

28. The method of claim 20, further comprising the step of measuring the ratio of LA to GLA in a sample of blood plasma phospholipids from said mammal, and adjusting the amount of GLA in said nutritional supplement to maintain said LA/GLA at or below 100.

29. The method of claim 20, further comprising the step of measuring the ratio of AA to DGLA in a sample of plasma phospholipids wherein an adequate level of a specific inhibitor of delta-5 desaturase is added to keep the AA/DGLA ratio less than 20 in the plasma phospholipids.

30. A food product comprising the nutritional supplement of claim 1.

31. The food product of claim 30, wherein said food product further comprises carbohydrate and protein.

32. The food product of claim 31, wherein the protein and the carbohydrate are present in a ratio of between 0.5 and 1.0 protein-to-carbohydrate, inclusive.

33. A method of preparing the nutritional supplement of claim 1, comprising the steps of:
(a) providing a source of fish oil comprising EPA and DHA;
(b) providing a source of GLA; and
(c) providing a source of a specific inhibitor of delta-5 desaturase enzymatic activity.

34. The method of claim 33, wherein said source of a specific inhibitor of delta-5 desaturase enzymatic activity is sesame oil comprising a lignan, and said method further comprises the step of crystallizing said lignan from said sesame oil.

35. The method of claim 36, wherein said crystallization step comprises the step of cystallizing said lignan in acetone solvent.
